(19) United States
(12) Patent Application Publication
(10) Pub. No.: US 2026/0171251 A1
(43) Pub. Date: Jun. 18, 2026

(54) PREDICTING CHANGES IN MEDICAL CONDITIONS USING MACHINE LEARNING MODELS

(71) Applicant: KONINKLIJKE PHILIPS N.V., EINDHOVEN (NL)

(72) Inventors: STEPHANIE LANIUS, ORLANDO, FL (US); ERINA GHOSH, ORLANDO, FL (US); LARY JAMES ESHELMAN, OSSINING, NY (US)

(21) Appl. No.: 19/532,289

(22) Filed: Feb. 6, 2026

Related U.S. Application Data
(63) Continuation of application No. 17/320,324, filed on May 14, 2021, now abandoned.
(60) Provisional application No. 63/042,781, filed on Jun. 23, 2020.

(51) Int. Cl. G16H 50/20; A61B 5/00; A61B 5/20; G06N 20/00; G16H 10/60; G16H 50/30

(52) U.S. Cl. CPC G16H 50/20; A61B 5/7267; A61B 5/7275; G06N 20/00; G16H 10/60; G16H 50/30; A61B 5/201

(57) ABSTRACT

Techniques are described herein for using time series data such as vital signs data and laboratory data or other time series data as input across machine learning models to predict a change in stage of a medical condition of a patient. In various embodiments, patient data comprising vital signs data of a patient and laboratory data or other time series data of the patient corresponding to an observation window may be received. A time series model may be used to predict a change in stage of a medical condition in the patient in a prediction window based on the patient data. The predicted change in stage of the medical condition may be output.

### CROSS REFERENCE TO RELATED APPLICATION

The present application is a continuation of co-pending U.S. patent application Ser. No. 17/320,324, filed May 14, 2021, which is a U.S. Utility application of U.S. Provisional Application Ser. No. 63/042,781 filed Jun. 23, 2020. These applications are hereby incorporated by reference herein.

### TECHNICAL FIELD

Various embodiments described herein are directed generally to health care and/or artificial intelligence. More particularly, but not exclusively, various methods and systems disclosed herein relate to using time series data as input across machine learning models to predict a change in a medical condition of a patient.

### BACKGROUND

Patients (e.g., in an intensive care unit of a hospital) may develop new medical conditions as secondary complications of critical illnesses. These new medical conditions may be caused by factors such as interventions and organ failures. For example, acute kidney injury (AKI) occurs in a significant cohort in the intensive care unit.

While guidelines may be used to determine a patient's current stage of a medical condition such as AKI, conventional algorithms used in a clinical setting are unable to predict a medical condition such as AKI in advance. Additionally, conventional algorithms developed by researchers typically use one value for each input and therefore are unable to capture information in trends in data and unable to accurately predict a medical condition such as AKI in advance. Without the ability to accurately predict a medical condition in advance, clinicians managing patients may not be able to take steps to prevent new medical conditions from developing or existing medical conditions from worsening and thereby improve patient outcomes such as mortality, length of stay, and post-discharge quality of life.

### SUMMARY

The present disclosure is directed to methods and systems for using time series data such as vital signs data and laboratory data as input across a machine learning model to predict a change in stage of a medical condition of a patient. For example, in various embodiments, the probability of a patient developing a medical condition or recovering from a medical condition such as AKI at a specified time window in the future (i.e., a prediction window) is predicted using a recurrent neural network (RNN) with long short-term memory (LSTM) units. In some implementations, a time series or array of values is used as input for each feature in a deep learning model, in order to learn from trends in data. In embodiments, patient data from an observation window is collected and used to predict the change in stage in the prediction window. Additionally, in embodiments, a gap window is provided between the observation window and the prediction window. The gap window may allow time for a clinician to take steps to react to the prediction.

In embodiments, an RNN with LSTM units leverages trend information from time series data inputs to predict whether a patient is likely to develop AKI or recover from AKI at a specified time window in the future. In particular, in embodiments, an RNN is used to predict an increase in AKI stage, a decrease in AKI stage, or no change in AKI stage. Additionally, in embodiments, missing clinical data of a patient (e.g., vital signs data and/or laboratory data) is imputed, to account for differing measurement frequencies among different data types (e.g., vital signs data may be measured on an hourly basis, while laboratory data may be measured on a daily basis). In embodiments, a length of an observation window may be varied to account for the measurement frequencies and/or availability of data. In embodiments, the parameters of the RNN-LSTM model including the loss function and error metrics are optimized to predict an increase in AKI stage and a decrease in AKI stage, as opposed to no change in AKI stage.

Generally, in one aspect, a method implemented using one or more processors may include: receiving patient data including time series data of a patient corresponding to an observation window; using a time series model to predict a change in stage of a medical condition in the patient in a prediction window based on the patient data; and outputting the predicted change in stage of the medical condition.

In various embodiments, the time series data of the patient includes vital signs data of the patient and laboratory data of the patient. In various embodiments, the time series model is trained using training data including training vital signs data and training laboratory data corresponding to training observation windows. In various embodiments, the training data is labeled with an increase in stage label, a decrease in stage label, or a no change in stage label, based on a change in stage of the medical condition in a training prediction window.

In various embodiments, the time series model is a recurrent neural network model with long short-term memory units. In various embodiments, the training the recurrent neural network model further includes using a binary cross-entropy loss function. In various embodiments, in the training of the time series model, a first penalty is assigned to incorrectly identifying the no change in stage label that is lower than a second penalty assigned to incorrectly identifying the increase in stage label and the decrease in stage label.

In various embodiments, the observation window and the prediction window are separated by a gap window that is longer than the prediction window. In various embodiments, a length of the observation window is determined based on a number of hours the patient has been hospitalized. In various embodiments, the medical condition is acute kidney injury.

In addition, in some implementations, computer program product may include one or more non-transitory computer-readable storage media having program instructions collectively stored on the one or more computer-readable storage media. The program instructions may be executable to: receive patient data including time series data of a patient corresponding to an observation window; use a time series model to predict a change in stage of a medical condition in the patient in a prediction window based on the patient data; and output the predicted change in stage of the medical condition.

In addition, in some implementations, a method implemented using one or more processors may include: receiving training data including time series data corresponding to an observation window, wherein the training data is labeled based on a change in stage of a medical condition in a prediction window; generating preprocessed training data using the training data by imputing missing values in the time series data; and training a time series model to predict the change in stage of the medical condition using the preprocessed training data, wherein the observation window and the prediction window are separated by a gap window that is longer than the prediction window.

In various embodiments, the generating the preprocessed training data further includes removing data corresponding to observation windows having time series data that fails to satisfy one or more criteria. In various embodiments, the preprocessed training data is a tensor with each sample containing an array of feature values over time. In various embodiments, the method further includes using adaptive boosting to identify, in the training data, important features for predicting the medical condition, and using the important features in the training the time series model.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 illustrates an example environment in which selected aspects of the present disclosure may be implemented.

FIG. 2 depicts an example method for practicing selected aspects of the present disclosure.

FIG. 3 depicts an example method for practicing selected aspects of the present disclosure.

FIG. 4 depicts one example of how a patient may be continuously assessed according to the method of FIG. 3.

FIG. 5 depicts another example of how a patient may be continuously assessed according to the method of FIG. 3.

FIG. 6 depicts one example of a data flow through a recurrent neural network according to aspects of the present disclosure.

FIG. 7 depicts an example computer architecture.

### DETAILED DESCRIPTION

Modern artificial intelligence (“AI”) techniques such as deep learning have numerous applications. While relatively adaptable across domains, these deep learning models may not be configured to predict a change in stage of a medical condition in a patient. Moreover, AI models that process time series data are more complex, less readily available, and even when available, are not easily adapted for new domains. In view of the foregoing, various embodiments and implementations of the present disclosure are directed to using time series data as input across machine learning models to predict a change in a medical condition of a patient.

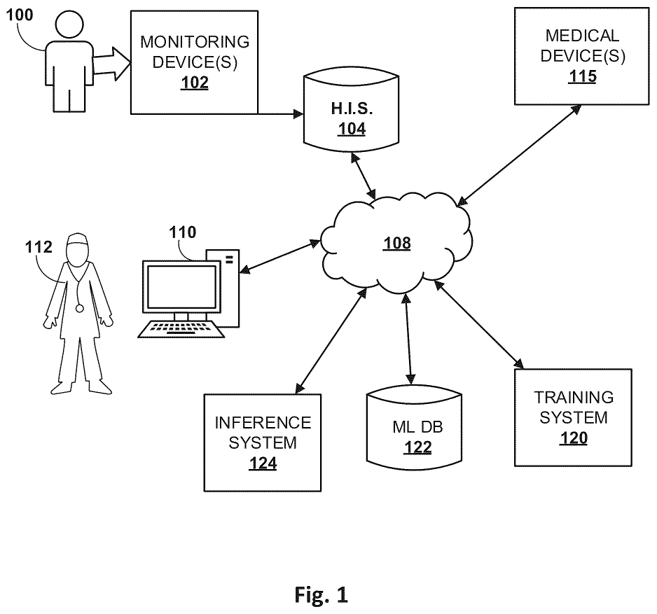

FIG. 1 depicts an example environment in which selected aspects of the present disclosure may be implemented, in accordance with various embodiments. The computing devices depicted in FIG. 1 may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a standalone interactive speaker (which in some cases may include a vision sensor), a smart appliance such as a smart television (or a standard television equipped with a networked dongle with automated assistant capabilities), and/or a wearable apparatus of the user that includes a computing device (e.g., a watch of the user having a computing device, glasses of the user having a computing device, a virtual or augmented reality computing device). Additional and/or alternative computing devices may be provided.

In FIG. 1, a patient 100 is being monitored by monitoring device(s) 102, e.g., at a hospital, to obtain time series data in the form of vital signs data of the patient 100. For example, this vital signs data may include body temperature data, blood pressure data, pulse (heart rate) data, breathing rate (respiratory rate) data, weight data, and/or any other health data collected from the patient 100 by the monitoring device(s) 102 as illustrated in FIG. 1. This vital signs data may be provided to and/or stored in a hospital information system (“HIS”) 104 or another similar healthcare system, e.g., as part of an electronic health record (“EHR”) for the patient 100. While the vital signs data is provided directly to HIS 104 in FIG. 1, this is not meant to be limiting. In various embodiments, the vital signs data may be provided to HIS 104 over one or more networks 108, which can include one or more local area networks and/or one or more wide area networks such as the Internet.

In FIG. 1, medical device(s) 115 may be a laboratory testing device such as a blood chemistry analyzer or any other type of device that performs laboratory testing, e.g., on blood samples or other samples collected from the patient 100, to obtain time series data in the form of laboratory data of the patient 100. For example, this laboratory data may include creatinine data, blood urea nitrogen (BUN) data, glucose data, lactate data, and/or any other health data of the patient 100 obtained through laboratory testing, e.g., on samples collected from the patient 100. In other implementations, the medical device(s) 115 may be a ventilator, infusion pump, dialysis machine, or any other type of medical device that measures, records, generates, and/or otherwise obtains time series data associated with the patient 100. This laboratory data or other time series data obtained by the medical device(s) 115 may be provided to and/or stored in HIS 104 or another similar healthcare system, e.g., as part of an EHR for the patient 100.

A training system 120 and an inference system 124 may be implemented using any combination of hardware and software in order to create, manage, and/or apply time series machine learning model(s) stored in a machine learning (“ML”) model database (“DB”) 122. In implementations, the machine learning model may be a recurrent neural network model. Training system 120 may be configured to apply training data such as vital signs data and laboratory data or other time series data corresponding to observation windows as input across one or more of the models in database 122 to generate output. The output generated using the training data may be compared to labels associated with prediction windows corresponding to the training data in order to determine error(s) associated with the model(s). A training example's label may indicate, for instance, a change in stage of a medical condition in a patient from which the training example was generated. The change in stage may be an increase in stage, a decrease in stage, or no change in stage. These error(s) may then be used, e.g., by training system 120, to train the model(s) using techniques such as back propagation and gradient descent (stochastic or otherwise).

Inference system 124 may be configured to use the trained machine learning model(s) in database 122 to infer changes in stage of medical conditions of patients based on patient data including vital signs data and laboratory data or other time series data using techniques described herein. In some embodiments, training system 120 and/or inference system 124 may be implemented as part of a distributed computing system that is sometimes referred to as the “cloud,” although this is not required.

FIG. 1 also depicts health care personnel such as a doctor 112 that operates a computing device 110 in order to make inferences about medical conditions of patients (e.g., the patient 100) as described herein. In particular, computing device 110 may be connected to network(s) 108 and thereby may interact with inference system 124 in order to make medical condition inferences as described herein. For example, the doctor 112 may be able to make inferences about a change in stage of a medical condition in the patient 100 based on the vital signs data of the patient 100 obtained by the monitoring device(s) 102 and the laboratory data or other time series data of the patient 100 obtained by the medical device(s) 115.

In some embodiments, the ability to make these inferences may be provided as part of a software application that aids doctor 112 with diagnosis, e.g., a clinical decision support (“CDS”) application. In some such embodiments, doctor 112 may rely on the inference to predict a medical condition or change in medical condition in advance and identify an opportunity to take mitigating steps and thereby improve a medical outcome for the patient 100. Alternatively, the inferences may be used by the doctor 112 to track the progress of a treatment for the medical condition to assure that the treatment and amount are appropriate. Additionally, the inferences may be used as a “second opinion” to buttress or challenge a medical opinion of the doctor 112.

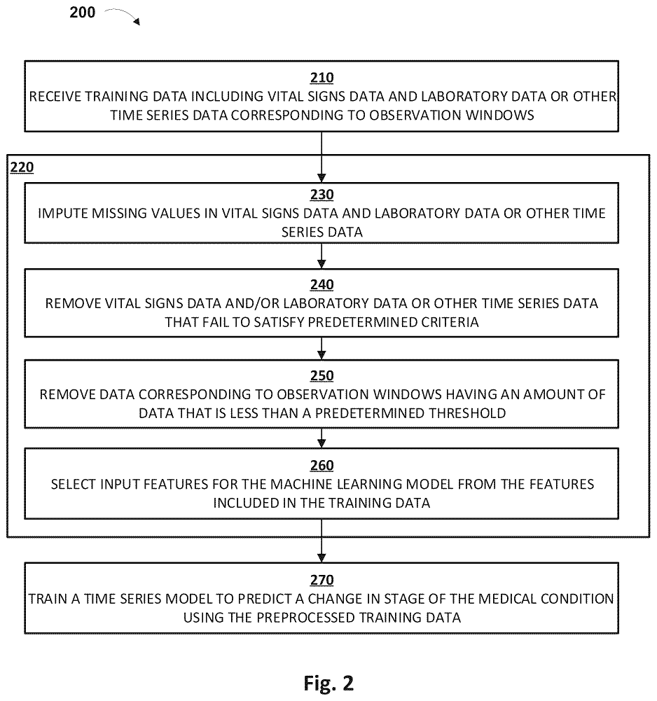

FIG. 2 illustrates a flowchart of an example method 200 for practicing selected aspects of the present disclosure. The operations of FIG. 2 can be performed by one or more processors, such as one or more processors of the various computing devices/systems described herein. For convenience, operations of method 200 will be described as being performed by a system configured with selected aspects of the present disclosure. Other implementations may include additional operations than those illustrated in FIG. 2, may perform step(s) of FIG. 2 in a different order and/or in parallel, and/or may omit one or more of the operations of FIG. 2.

At block 210, the system may receive training data including vital signs data and laboratory data or other time series data corresponding to observation windows. In implementations, block 210 comprises the training system 120 receiving training data for a machine learning model, the training data including vital signs data and laboratory data or other time series data corresponding to observation windows from HIS 104 or another data source (not shown). In embodiments, the vital signs data may include body temperature data, blood pressure data, pulse (heart rate) data, breathing rate (respiratory rate) data, weight data, and/or any other health data collected from patients. In embodiments, the laboratory data may include creatinine data, blood urea nitrogen (BUN) data, glucose data, lactate data, and/or any other health data of patients obtained through laboratory testing of patients. In embodiments, the other time series data may include time series data obtained from a ventilator, infusion pump, dialysis machine, or any other type of medical device. In embodiments, the training data that is received at block 210 is labeled based on a change in stage of a medical condition (e.g., AKI) in a prediction window.

Still referring to block 210, in embodiments, the training data includes samples grouped into three groups, i.e., increase in stage (deterioration) of a medical condition, decrease in stage (improvement) of a medical condition, and no change in stage of a medical condition. In an example in which the medical condition is AKI, the AKI stage may be one of three values (1, 2, and 3). An improvement in kidney function may be characterized as a decrease in stage of AKI, a deterioration in kidney function may be characterized as an increase in stage of AKI, and unchanged kidney function may be characterized as no change in stage of AKI. In an example set of training data, there are few changes in stage, and 88% of samples belong to the no change group.

In embodiments, the training data that is received at block 210 may be sets of time series data including vital signs data and laboratory data or other time series data collected from patients during four-hour observation windows. In embodiments, the training data that is received at block 210 may be labeled based on changes in stage of a medical condition of the patients during four-hour prediction windows. In embodiments, the observation windows and the prediction windows are separated by a six-hour gap window. In embodiments, the lengths of the observation windows, gap windows, and prediction windows are configurable (e.g., by the doctor 112), and the above-mentioned lengths are not limiting. In implementations, the length of the observation window may be variable based on a number of hours the patient has been hospitalized.

In embodiments, the length of the gap window may be set to allow the doctor 112 time to react to a predicted change in stage of a medical condition in a patient. For example, in response to a prediction that a medical condition will increase in stage in six hours (i.e., after the gap window), the doctor 112 may take measures to attempt to prevent (or ease) this deterioration. In this example, to identify and implement those measures, the doctor 112 may need a certain amount of gap or lead time. In this example, the doctor 112 may choose and implement the measures within the time corresponding to the gap window, based on a prediction made using patient data (e.g., vital signs data and laboratory data or other time series data) obtained during the observation window.

Still referring to FIG. 2, at block 220, which includes blocks 230 to 260, the system may generate preprocessed training data using the training data received at block 210. At block 230, the system may impute missing values in the vital signs data and the laboratory data or other time series data. In implementations, block 230 comprises the training system 120 imputing missing values in the vital signs data and the laboratory data or other time series data included in the training data received at block 210. In embodiments, the vital signs data and/or the laboratory data or the other time series data may be irregularly sampled and therefore different features (i.e., different types of vital signs data and/or different types of laboratory data or other time series data) may be missing at different time points in the training data received at block 210. In an example, the training data may be time series data including hourly samples, and different types of vital signs data and/or laboratory data or other time series data may be missing from various hourly samples (i.e., at various time points) in the training data. In implementations, the training system 120 may impute values for these missing features.

Still referring to block 230, in implementations, the training system 120 may impute missing values for a type of vital signs data from past values when that type of vital signs data was last measured within a first predetermined time period, and the training system 120 may impute missing values for a type of laboratory data or other time series data from past values when that type of laboratory data or other time series data was last measured within a second predetermined time period. In implementations, the last measurement for a type of data may be used as the imputed value for that type of data for a time point at which a measurement is missing. In other implementations, for a time point at which a measurement is missing, an imputed value may be determined using the last measurement for that type of data based on predetermined rules. In implementations, for a particular time point, when the last measurement of a type of vital signs data was not within the first predetermined time period or the last measurement of a type of laboratory data or other time series data was not within the second predetermined time period, the training system 120 may avoid imputing a missing value for that particular time point. In other implementations, a different predetermined time period may be used for each type of vital signs data and for each type of laboratory data.

Still referring to block 230, in an example, values for missing types of laboratory data may be imputed from past values for up to 26 hours. In particular, in the example, if a measurement is not available for a type of laboratory data (e.g., creatinine data) for a particular time point in an observation window, then the last measurement for that type of laboratory data may be used for the particular time point as the imputed value, provided that the particular time point is within 26 hours of a time point corresponding to the last measurement. In other implementations, an imputed value may be determined using the last measurement for that type of vital laboratory data based on predetermined rules. Additionally, in an example, values for vital signs data may be imputed from past values for up to two hours. In particular, in the example, if a measurement is not available for a type of vital signs data (e.g., heart rate data) for a particular time point in an observation window, then the last measurement for that type of vital signs data may be used for the particular time point as the imputed value, provided that the particular time point is within two hours of a time point corresponding to the last measurement. In other implementations, an imputed value may be derived from the last measurement for that type of vital signs data based on predetermined rules.

Still referring to FIG. 2, at block 240, the system may remove types of vital signs data and/or types of laboratory data or other time series data included in the training data that fail to satisfy predetermined criteria. In implementations, block 240 comprises the training system 120 removing types of vital signs data and/or types of laboratory data or other time series data included in the training data received at block 210 that fail to satisfy predetermined criteria. In implementations, the predetermined criteria include a maximum acceptable amount of missing data per feature (e.g., per type of vital signs data and laboratory data or other time series data). The maximum acceptable amount of missing data may be different for each feature in the training data and may be evaluated after imputing the missing values at block 230. In other implementations, the maximum acceptable amount of missing data may be evaluated prior to imputing the missing values at block 230. In response to the amount of missing data of a particular feature exceeding the predetermined criteria including the maximum acceptable amount of missing data per feature, the training system 120 may remove the data corresponding to the particular feature from the training data.

Still referring to block 240, in an example, the maximum acceptable amount of missing data may be 50% for creatinine data. If creatinine data is missing for more than 50% of the time points in the training data, then the training system 120 may remove the creatinine data from the training data. On the other hand, if creatine data is not missing for more than 50% of the time points in the training data, then the training system 120 may retain the creatinine data in the training data. In this manner, the training system 120 may remove features that are infrequently measured from the features that are used as inputs to the machine learning model.

Still referring to block 240, in implementations, the training system 120 may use other predetermined criteria instead of or in addition to the maximum acceptable amount of missing data per feature. In an example, other predetermined criteria used by the training system 120 may include quality criteria that assess the quality of the data per feature.

Still referring to FIG. 2, at block 250, the system may remove data corresponding to observation windows having an amount of data that is less than a predetermined threshold. In implementations, block 250 comprises the training system 120 identifying observation windows that are associated with an amount of data that is less than a predetermined threshold and removing the identified observation windows from the training data. In an example, the predetermined threshold is at least three data points for at least half of the features in a six-hour observation window with hourly sampling. In implementations, this predetermined threshold may be configurable based on the availability of the data and the clinical application (e.g., a particular medical condition for which a change is being predicted).

Still referring to FIG. 2, at block 260, the system may select input features for the machine learning model from the features included in the training data. In implementations, block 260 comprises the training system 120 selecting input features for the machine learning model from the features included in the training data. In some implementations, all of the types of data remaining in the training data (i.e., after any types of data are removed at block 240) are selected as features to be used as inputs across the machine learning model. In other implementations, the training system 120 may use a second machine learning model to identify predictive features in the training data and select the identified features to be used as inputs across the machine learning model. In implementations, adaptive boosting algorithms such as AdaBoost and/or BagBoost may be used to train the second machine learning model to make a yes or no prediction regarding the existence of a medical condition (e.g., AKI) in a patient at a time that is six hours after the time when the prediction is made. The training system 120 then selects the features (e.g., particular types of vital signs data and laboratory data or other time series data) identified as predictive by this second machine learning model as features to be used as inputs across the machine learning model.

Still referring to FIG. 2, at block 270, the system may train a time series model to predict a change in stage of the medical condition using the preprocessed training data. In implementations, block 270 comprises the training system 120 training a machine learning model to predict the change in stage of the medical condition using the preprocessed training data generated at block 220. In implementations, the machine learning model may be a recurrent neural network. In implementations, the training data corresponding to the features selected to be used as inputs across the machine learning model at block 260 are saved as a tensor with each sample containing an array of feature values over time.

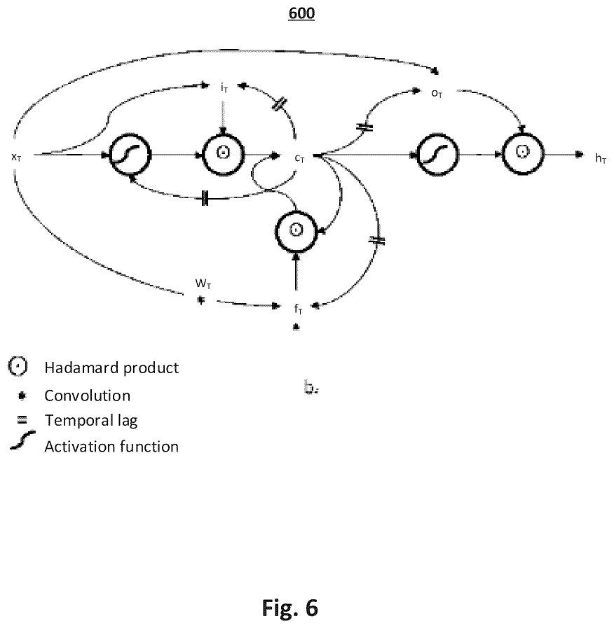

Still referring to block 270, in implementations, the training data is then loaded in batches and used to train the machine learning model, which may be a single layer LSTM recurrent neural network with input and forget gates, as illustrated in FIG. 6. The time series training data is passed through the network in a sequential manner. In implementations, the network for each time point uses the data at the time point and the state of the network at the previous time point modulated by the forget gate. In this manner, the machine learning model is trained such that weight matrices are learned for each node.

Still referring to block 270, in implementations, there may be a large class imbalance in the training data. For example, in the training data, a relatively larger number of the samples may belong to the no change in stage of a medical condition group, and a relatively smaller number of samples may belong to the increase in stage of a medical condition group or decrease in stage of a medical condition group. In implementations, the training system 120 trains the machine learning model to predict the increase in stage or the decrease in stage in the prediction window based on the observation window data by optimizing the error matrix and assigning a relatively lower penalty for incorrectly identifying the no change label and a relatively higher penalty for incorrectly identifying the increase in stage or decrease in stage labels. In implementations, the penalty for incorrectly identifying the increase in stage may be the same as the penalty for incorrectly identifying the decrease in stage. In implementations, the training system 120 uses a binary cross-entropy loss function in training the machine learning model. The training system 120 may train the machine learning model for multiple epochs, and the training system 120 may evaluate the performance of the machine learning model in the training data as well as additional test data.

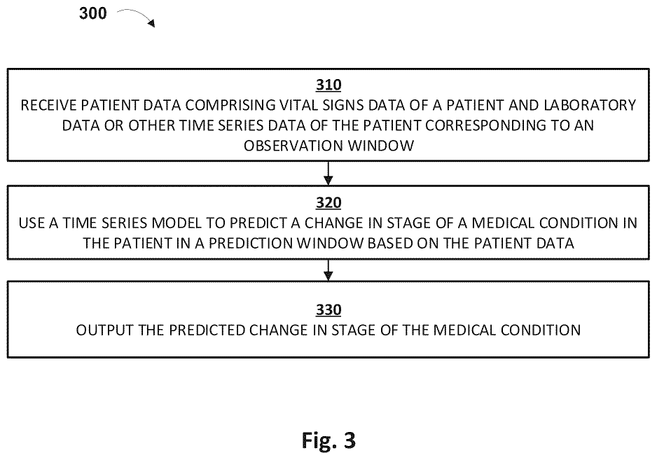

FIG. 3 illustrates a flowchart of an example method 300 for practicing selected aspects of the present disclosure. The operations of FIG. 3 can be performed by one or more processors, such as one or more processors of the various computing devices/systems described herein. For convenience, operations of method 300 will be described as being performed by a system configured with selected aspects of the present disclosure. Other implementations may include additional operations than those illustrated in FIG. 3, may perform step(s) of FIG. 3 in a different order and/or in parallel, and/or may omit one or more of the operations of FIG. 3.

At block 310, the system may receive patient data comprising vital signs data of a patient and laboratory data or other time series data of the patient corresponding to an observation window. In implementations, block 310 comprises the inference system 124 receiving vital signs data of a patient 100 from the monitoring device(s) 102 (e.g., via HIS 104) and receiving laboratory data or other time series data of the patient 100 from the medical device(s) 115 (e.g., via HIS 104). The vital signs data and the laboratory data or other time series data may be collected during an observation window. In an example, the observation window may be four hours in length.

Still referring to block 310, in embodiments, the vital signs data may include body temperature data, blood pressure data, pulse (heart rate) data, breathing rate (respiratory rate) data, weight data, and/or any other health data collected from the patient 100. In embodiments, the laboratory data may include creatinine data, blood urea nitrogen (BUN) data, glucose data, lactate data, and/or any other health data of the patient 100 obtained through laboratory testing samples collected from the patient 100. In embodiments, the other time series data may include time series data of the patient 100 obtained from a ventilator, infusion pump, dialysis machine, or any other type of medical device. In embodiments, the inference system 124 may receive types of vital signs data and types of laboratory data or other time series data selected to be used as inputs at block 260 of FIG. 2.

Still referring to FIG. 3, at block 320, the system may use a time series model to predict a change in stage of a medical condition in the patient in a prediction window based on the patient data. In implementations, block 320 comprises the inference system 124 using a recurrent neural network model trained according to the method of FIG. 2 to predict a change in stage of a medical condition in the patient 100 in a prediction window based on the patient data received at block 310. In particular, in implementations, the inference system 124 may use the vital signs data and the laboratory data or other time series data included in the patient data received at block 310 as inputs across the machine learning model trained at block 270 of FIG. 2. The inference system 124 may then receive as an output of the machine learning model one of the increase in stage label, the decrease in stage label, or the no change in stage label, indicating a predicted change in stage of a medical condition of the patient 100.

Still referring to FIG. 3, at block 330, the system may output the predicted change in stage of the medical condition. In implementations, block 320 comprises the inference system 124 outputting the change in stage of the medical condition of the patient 100 that was predicted at block 320. In particular, in implementations, the inference system 124 may output the predicted change in stage of the medical condition to the computing device 110. The computing device 110 may include a software application such as a CDS application, and the CDS application of the computing device 110 may receive the output of the predicted change in stage of the medical condition of the patient 100 and display the predicted change in stage of the medical condition using a graphical user interface provided by the software application. A doctor 112 using the computing device 110 may then review the predicted change in stage of the medical condition that is displayed within a graphical user interface provided by the software application. In embodiments, the method of FIG. 3 may be repeated at predetermined intervals, e.g., every x hours, where x is the length of the prediction window.

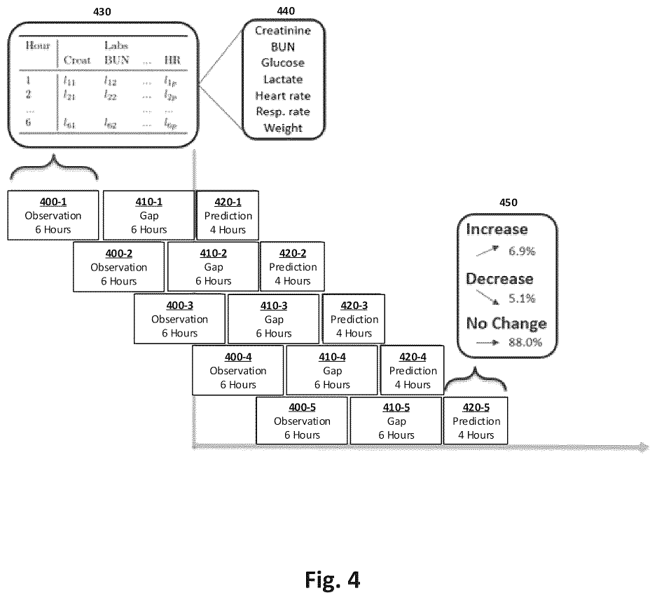

FIG. 4 depicts an example of assessing a patient continuously according to the method of FIG. 3. In particular, as illustrated in FIG. 4, hourly continuous data 430 for a plurality of features 440 are collected in observation windows 400-1, 400-2, 400-3, 400-4, 400-5 and used as inputs into a recurrent neural network that is used to predict a change in stage 450 of a medical condition in a patient in prediction windows 420-1, 420-2, 420-3, 420-4, 420-5. In the example illustrated in FIG. 4, the prediction windows 420-1, 420-2, 420-3, 420-4, 420-5 are separated from the observation windows 400-1, 400-2, 400-3, 400-4, 400-5 by gap windows 410-1, 410-2, 410-3, 410-4, 410-5 that are longer in duration than the prediction windows 420-1, 420-2, 420-3, 420-4, 420-5.

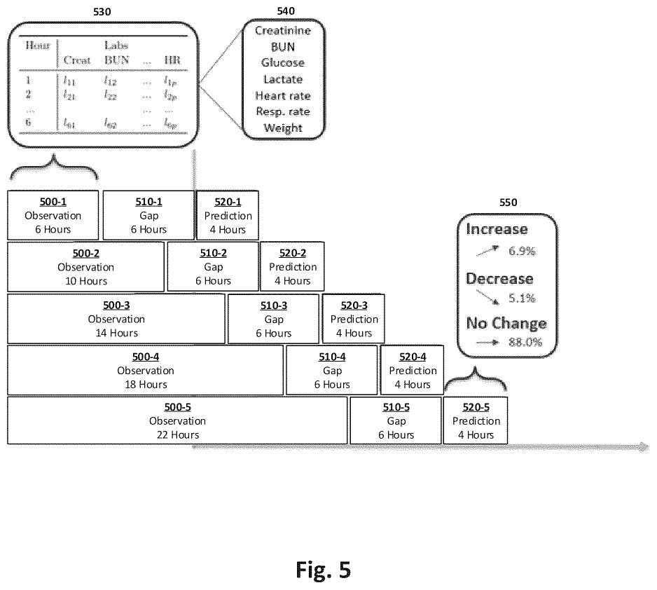

FIG. 5 depicts another example of assessing a patient continuously according to the method of FIG. 3. In particular, as illustrated in FIG. 5, hourly continuous data 530 for a plurality of features 540 are collected in observation windows 500-1, 500-2, 500-3, 500-4, 500-5 and used as inputs into a recurrent neural network that is used to predict a change in stage 550 of a medical condition in a patient in prediction windows 520-1, 520-2, 520-3, 520-4, 520-5. In the example illustrated in FIG. 5, the prediction windows 520-1, 520-2, 520-3, 520-4, 520-5 are separated from the observation windows 500-1, 500-2, 500-3, 500-4, 500-5 by gap windows 510-1, 510-2, 510-3, 510-4, 510-5 that are longer in duration than the prediction windows 520-1, 520-2, 520-3, 520-4, 520-5. In the example illustrated in FIG. 5, the observation windows 500-1, 500-2, 500-3, 500-4, 500-5 vary in length based upon a length of time the patient has been hospitalized.

Still referring to FIG. 5, in implementations, all patient data including vital signs data and laboratory data or other time series data collected in the observation windows 500-1, 500-2, 500-3, 500-4, 500-5 are used to predict the change in stage of a medical condition in the prediction windows 520-1, 520-2, 520-3, 520-4, 520-5 using the recurrent neural network. Due to the use of a forget gate in the recurrent neural network, in the observation window, vital signs data and laboratory data or other time series data collected closer to the end of the observation windows 500-1, 500-2, 500-3, 500-4, 500-5 have a greater influence on the prediction than vital signs data and laboratory data or other time series data collected closer to the beginning of the observation windows 500-1, 500-2, 500-3, 500-4, 500-5.

FIG. 6 depicts an example of a data flow 600 through the recurrent neural network with LSTM units that is trained according to the method of FIG. 3 and used to predict a change in stage of a medical condition according to the method of FIG. 4. In implementations, in the data flow 600, patient data including vital signs data and laboratory data or other time series data (x_{T}) enters the neural network, flows through a normalizing activation function, and is “multiplied” with the parameters of the input gate (i_{T}). The inner state (c_{T}) then flows back to itself (though f_{T}), so C_{T-1} influences c_{T}. The output (h_{T}) is dependent on c_{T} and o_{T}, which are parameters of the output gate.

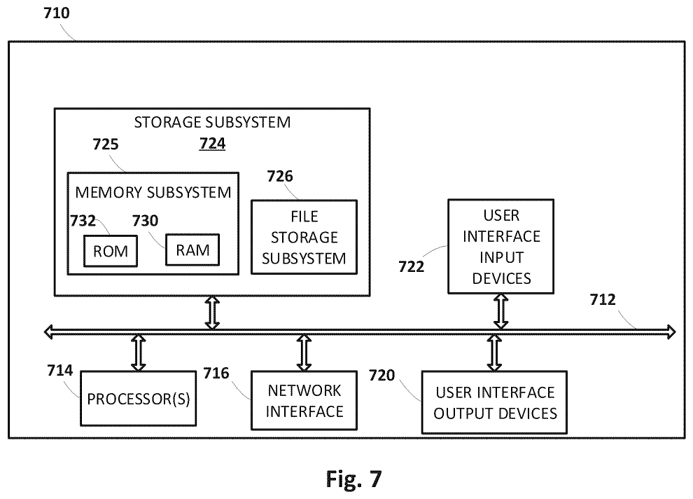

FIG. 7 is a block diagram of an example computing device 710 that may optionally be utilized to perform one or more aspects of techniques described herein. Computing device 710 typically includes at least one processor 714 which communicates with a number of peripheral devices via bus subsystem 712. These peripheral devices may include a storage subsystem 724, including, for example, a memory subsystem 725 and a file storage subsystem 726, user interface output devices 720, user interface input devices 722, and a network interface subsystem 716. The input and output devices allow user interaction with computing device 710. Network interface subsystem 716 provides an interface to outside networks and is coupled to corresponding interface devices in other computing devices.

User interface input devices 722 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 710 or onto a communication network.

User interface output devices 720 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 710 to the user or to another machine or computing device.

Storage subsystem 724 stores programming and data constructs that provide the functionality of some or all of the modules described herein. For example, the storage subsystem 724 may include the logic to perform selected aspects of the methods of FIGS. 2 and 3, as well as to implement various components depicted in FIG. 1.

These software modules are generally executed by processor 714 alone or in combination with other processors. Memory subsystem 725 included in the storage subsystem 724 can include a number of memories including a main random access memory (RAM) 730 for storage of instructions and data during program execution and a read only memory (ROM) 732 in which fixed instructions are stored. A file storage subsystem 726 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 726 in the storage subsystem 724, or in other machines accessible by the processor(s) 714.

Bus subsystem 712 provides a mechanism for letting the various components and subsystems of computing device 710 communicate with each other as intended. Although bus subsystem 712 is shown schematically as a single bus, alternative implementations of the bus subsystem may use multiple busses.

Computing device 710 can be of varying types including a workstation, server, computing cluster, blade server, server farm, or any other data processing system or computing device. Due to the ever-changing nature of computers and networks, the description of computing device 710 depicted in FIG. 7 is intended only as a specific example for purposes of illustrating some implementations. Many other configurations of computing device 710 are possible having more or fewer components than the computing device depicted in FIG. 7.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1-15. (canceled)

16. A method for assessing a change in a stage of a medical condition of a patient, the method comprising:
obtaining patient data comprising time series data of the patient corresponding to an observation window;
predicting, by a time series model based on the patient data, a change in the stage of the medical condition in a prediction window and a period for a clinician to take one or more steps to react to the predicted change in a gap window, wherein:
the time series model is trained using training data corresponding to training observation windows, the training data comprising at least one of training vital signs data or training laboratory data, and
the time series model is trained by assigning a first penalty for incorrectly identifying a no change in the stage of the medical condition and assigning a second penalty for incorrectly identifying an increase in the stage and a decrease in the stage, the second penalty being higher than the first penalty; and
outputting the prediction window and the gap window.

17. The method of claim 16, wherein:
the training data is labeled with an increase in stage label, a decrease in stage label, or a no change in stage label, based on a change in a stage of the medical condition in a training prediction window, and
the time series model is trained by assigning the first penalty for incorrectly identifying the no change in stage label and assigning a second penalty for incorrectly identifying the increase in stage label and the decrease in stage label.

18. The method of claim 16, wherein the observation window and the prediction window are separated by the gap window.

19. The method of claim 16, wherein:
the time series model is a recurrent neural network model with long short-term memory, and
the recurrent neural network model is trained using a binary cross-entropy loss function.

20. The method of claim 16, wherein a length of the observation window is determined based on a number of hours the patient has been hospitalized.

21. The method of claim 16, wherein the medical condition is acute kidney injury.

22. A system for assessing a change in a stage of a medical condition of a patient, the system comprising:
a processor and memory, the processor configured to:
obtain patient data comprising time series data of the patient corresponding to an observation window;
predict, by a time series model based on the patient data, a change in the stage of the medical condition in a prediction window and a period for a clinician to take one or more steps to react to the predicted change in a gap window, wherein:
the time series model is trained using training data corresponding to training observation windows, the training data comprising at least one of training vital signs data or training laboratory data, and
the time series model is trained by assigning a first penalty for incorrectly identifying a no change in the stage of the medical condition and assigning a second penalty for incorrectly identifying an increase in the stage and a decrease in the stage, the second penalty being higher than the first penalty; and
output the prediction window and the gap window.

23. The system of claim 22, wherein:
the training data is labeled with an increase in stage label, a decrease in stage label, or a no change in stage label, based on a change in a stage of the medical condition in a training prediction window.

24. The system of claim 23, wherein:
the time series model is trained by assigning the first penalty for incorrectly identifying the no change in stage label and assigning a second penalty for incorrectly identifying the increase in stage label and the decrease in stage label.

25. The system of claim 22, wherein the observation window and the prediction window are separated by the gap window.

26. The system of claim 22, wherein:
the time series model is a recurrent neural network model with long short-term memory, and
the recurrent neural network model is trained using a binary cross-entropy loss function.

27. The system of claim 22, wherein the processor is further configured to determine the length of the observation window based on a number of hours the patient has been hospitalized.

28. The system of claim 22, wherein the medical condition is acute kidney injury.

29. A non-transitory computer-readable storage medium having stored a computer program comprising instructions, which, when executed by a processor, cause the processor to:
obtain patient data comprising time series data of the patient corresponding to an observation window;
predict, by a time series model based on the patient data, a change in the stage of the medical condition in a prediction window and a period for a clinician to take one or more steps to react to the predicted change in a gap window, wherein:
the time series model is trained using training data corresponding to training observation windows, the training data comprising at least one of training vital signs data or training laboratory data, and
the time series model is trained based on assigning a first penalty for incorrectly identifying a no change in the stage of the medical condition and assigning a second penalty for incorrectly identifying an increase in the stage and a decrease in the stage, the second penalty being higher than the first penalty; and
output the prediction window and the gap window.

30. The non-transitory computer-readable storage medium of claim 29, wherein:
the training data is labeled with an increase in stage label, a decrease in stage label, or a no change in stage label, based on a change in a stage of the medical condition in a training prediction window.

31. The non-transitory computer-readable storage medium of claim 30, wherein:
the time series model is trained by assigning the first penalty for incorrectly identifying the no change in stage label and assigning a second penalty for incorrectly identifying the increase in stage label and the decrease in stage label.

32. The non-transitory computer-readable storage medium of claim 29, wherein the observation window and the prediction window are separated by the gap window.

33. The non-transitory computer-readable storage medium of claim 29, wherein:
the time series model is a recurrent neural network model with long short-term memory, and
the recurrent neural network model is trained using a binary cross-entropy loss function.

34. The non-transitory computer-readable storage medium of claim 29, wherein the instructions, when executed by the processor, further cause the processor to determine the length of the observation window based on a number of hours the patient has been hospitalized.

35. The non-transitory computer-readable storage medium of claim 29, wherein the medical condition is acute kidney injury.
